(19) United States
(12) Patent Application Publication
(10) Pub. No.: US 2018/0000725 A1
(43) Pub. Date: Jan. 4, 2018

(54) ORALLY-DISSOLVING FILMS FOR DRUG DELIVERY THROUGH THE ORAL CAVITY

(71) Applicant: The Chinese University of Hong Kong, Hong Kong (CN)

(72) Inventors: Wai Yip LEE, Hong Kong (CN); Yuan FANG, Beijing (CN); Qingqing LI, LingBao City (CN); Hao HAN, Guangzhou (Canton) (CN); Ka Lun LAI, Hong Kong (CN)

(21) Appl. No.: 15/621,853

(22) Filed: Jun. 13, 2017

Related U.S. Application Data
(60) Provisional application No. 62/356,274, filed on Jun. 29, 2016.

(51) Int. Cl. A61K 9/00; A61K 31/12; A61K 31/404; A61K 31/4422

(52) U.S. Cl. CPC A61K 9/006; A61K 31/4422; A61K 31/12; A61K 31/404

(57) ABSTRACT

Orally dissolving films including a pharmaceutically active ingredient having a low bioavailability due to first-pass metabolism and/or low water solubility upon oral administration and a pharmaceutically acceptable excipient, and a medical application thereof.

### CROSS REFERENCE TO RELATED APPLICATION

This application claims priority to U.S. Provisional Patent Application No. 62/356,274 filed Jun. 29, 2016, the entire content of which are incorporated herein by reference in its entirety.

### TECHNICAL FIELD

The present application relates to the field of medicine, especially the field of a pharmaceutical formulation. More particularly, disclosed herein are orally dissolving films for drug delivery through the oral cavity to the systemic circulation and a medical application thereof.

### TECHNICAL BACKGROUND

Ropinirole (ROP), a non-ergoline type dopamine D2 receptor agonist, is used for treating Parkinson's disease (PD). Ropinirole is a highly water-soluble compound (133 mg/mL). After oral administration, it has a rapid absorption (Tₘₐₓ˜1.5 h) and also exhibits relatively low protein binding (10-39%) in the plasma due to its hydrophilicity.

However, ropinirole undergoes extensive metabolism in the liver via cytochrome P450 enzyme CYP1A2 in humans. Indeed, a low oral bioavailability of ropinirole (about 6 hours) is likely to be attributed to extensive first-pass metabolism and only 10% of an oral dose would be excreted unchanged. As a result, frequent administration of ropinirole is necessary (3 times a day), which may negatively impact patient compliance. Taking a tablet or capsule with a glass of water appears to be simple and easy for a normal person, but this activity poses a tremendous challenge to PD patients who are suffering from tremor, rigidity and limited mobility, especially during “off-periods”. As a result, there is a pressing need to develop formulations that enable simple and easy administration and fast drug action, as well as avoidance of first-pass metabolism, to improve bioavailability.

Nifedipine (NIF) is a model drug to treat hypertension and angina pectoris. Nifedipine has poor water-solubility that varies from 5 to 11 μg/ml under physiological conditions. All the current marketed products of nifedipine are oral dosage forms (such as oral tablets, soft gelatin capsules etc.) and can be administered at 5 or 10 mg (3 times a day) with a maximum dosage of 60 mg per day. Nifedipine is absorbed completely from the gastrointestinal tract (GI tract) after oral administration with a short Tₘₐₓ (1.6 to 4.2 hours), relative short half-life time (t_{1/2}, 2-5 hours) and a minimum effective plasma concentration at about 13.4 ng/ml.

However, the relative bioavailability of nifedipine from oral dosage forms (oral administration compared with intravenous administration) is relatively low (about 43.8%), which is due to its poor solubility and extensive first-pass metabolism. It is a well-known fact that nifedipine undergoes oxidative Metabolism via cytochrome P450 (CYP450) in the intestinal wall or liver. The use of nifedipine has also been proposed in the case of urgent situations, for example, in the management of angina attacks and/or hypertensive emergency. As a result, formulations of nifedipine that can provide a convenient means of administration and a fast drug action are highly desirable.

In addition, poor solubility is a major challenge affecting oral drug delivery. Amorphous solid dispersion is an emerging technology to overcome the solubility challenges of poorly soluble drugs. However, the delivery challenges of drug molecules appear to be multi-factorial. In addition to the poor solubility, extensive first-pass metabolism may also lead to poor oral bioavailability. As a result, amorphous solid dispersions alone may not necessarily provide a solution for improving oral bioavailability of poorly soluble drugs which undergo extensive first-pass metabolism. In light of the above limitations, there is a pressing need to develop new dosage forms to overcome multiple delivery challenges concurrently, which allow simple, easy and convenient administration, rapid onset of action, an increase in water-solubility and dissolution property as well as avoidance of first-pass metabolism to improve the bioavailability, especially the oral bioavailability.

### SUMMARY OF THE INVENTION

In one aspect, the present application provides an orally dissolving film (ODF) comprising a pharmaceutically active ingredient and a pharmaceutically acceptable excipient.

In another aspect, the present application provides a method for preparing an orally dissolving film, comprising dissolving a pharmaceutically active ingredient and a pharmaceutically acceptable excipient in an organic solvent or a mixed organic solvent to obtain a solution, applying the solution to form a film, and drying the film.

In another aspect, the present application provides a method for treating a disease, disorder and/or condition, comprising administering to a subject in need thereof an orally dissolving film comprising a pharmaceutically active ingredient and a pharmaceutically acceptable excipient.

In some embodiments of the present application, the pharmaceutically active ingredient has a low bioavailability due to first-pass metabolism and/or low water solubility upon oral administration.

In some embodiments of the present application, the pharmaceutically acceptable excipient is one or more selected from the group consisting of polyvinyl alcohol(s), ethylene-vinyl acetate copolymer(s), acrylic copolymer(s), cellulose(s) and derivative(s) thereof, polyvinylpyrrolidone(s), silicon rubber(s), polylactic acid(s), polyethylene glycol(s), surfactant(s), plasticizer(s), filler(s), coloring agent(s), and release agent(s).

In some embodiments of the present application, the pharmaceutically active ingredient is nifedipine, ropinirole, or curcumin.

In some embodiments of the present application, the pharmaceutically acceptable excipient is one or more selected from the group consisting of cellulose(s) and derivative(s) thereof, polyethylene glycol(s), and surfactant(s).

In some embodiments of the present application, the orally dissolving film is administered to the subject by sublingual and/or buccal administration route.

### BRIEF DESCRIPTION OF THE DRAWINGS

FIG. 1(a) shows the appearance of a typical orally dissolving film of ropinirole (ROP-ODF).

FIG. 1(b) is a microscopic picture of ROP-ODF.

FIG. 1(c) shows the appearance of a dissolving ROP-ODF.

FIG. 2 shows DSC thermograms of ropinirole, physical mixtures of ropinirole and a blank film, and ROP-ODF.

FIG. 3 shows the physical stability of ROP-ODF.

FIG. 4 shows comparison of the plasma profiles of ropinirole after sublingual and buccal administration of ROP-ODFs and gastric gavage of ropinirole at 1.5 mg/kg in rabbits (average±SEM, n=4, for oral gavage, n=2).

FIG. 5(a) shows the appearance of a typical orally dissolving film of nifedipine (NIF-ODF).

FIG. 5(b) shows the appearance of a dissolving NIF-ODF.

FIG. 6 shows XRPD patterns of NIF-ODF, nifedipine neat drug and physical mixtures.

FIG. 7 shows FTIR spectra of nifedipine neat drug, a blank film, a physical mixture of nifedipine and a blank film, and NIF-ODF.

FIG. 8 shows the physical stability of NIF-ODF (*statistically significant, p<0.05).

FIG. 9 shows comparison of the plasma profiles of nifedipine following sublingual and buccal administration of NIF-ODFs and oral administration of nifedipine in rabbits (average±SEM, n=4, for oral gavage, n=3).

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc.

Unless the context requires otherwise, throughout the specification and claims which follow, the word “comprise” and variations thereof, such as, “comprises” and “comprising” are to be construed in an open, inclusive sense, which is as “including, but not limited to”.

Reference throughout this specification to “one embodiment”, or “an embodiment”, or “in another embodiment”, or “some embodiments”, or “in some embodiments” means that a particular referent feature, structure, or characteristic described in connection with the embodiment is included in at least one embodiment. Thus, the appearance of the phrases “In some embodiments of the present application”, or “in an embodiment”, or “in another embodiment”, or “in some embodiments” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a”, “an”, and “the” include plural referents unless the content clearly dictates otherwise. In this application, the use of “or” means “and/or” unless stated otherwise.

In one aspect, the present application provides an orally dissolving film comprising a pharmaceutically active ingredient and a pharmaceutically acceptable excipient.

In some embodiments of the present application, the pharmaceutically active ingredient has a low bioavailability due to first-pass metabolism and/or low water solubility upon oral administration, preferably nifedipine, ropinirole and curcumin.

In some embodiments of the present application, the pharmaceutically active ingredient can be water-soluble or water-insoluble, and has a low bioavailability due to first-pass metabolism or low water solubility or the both upon oral administration.

In some embodiments of the present application, the pharmaceutically active ingredient in the orally dissolving film of the present application is amorphous, partially crystalline or crystalline, preferably amorphous.

In some embodiments of the present application, the pharmaceutically acceptable excipient is one or more selected from the group consisting of film-forming material(s), adhesive(s), lubricant(s), surfactant(s), plasticizer(s), filler(s), coloring agent(s), and release agent(s).

In some embodiments of the present application, the pharmaceutically acceptable excipient is one or more selected from the group consisting of polyvinyl alcohol(s), ethylene-vinyl acetate copolymer(s), acrylic copolymer(s) (such as methacrylate-methylacrylic acid copolymer), cellulose(s) and derivative(s) thereof (such as hydroxypropyl methyl cellulose, hydroxypropyl cellulose, methylcellulose, ethylcellulose and the like), polyvinylpyrrolidone(s), silicon rubber(s), polylactic acid(s), polyethylene glycol(s), surfactant(s), plasticizer(s), filler(s), coloring agent(s), and release agent(s).

In some embodiments of the present application, the pharmaceutically acceptable excipient is one or more selected from the group consisting of cellulose(s) and derivative(s) thereof, polyethylene glycol(s), and surfactant(s).

In some embodiments of the present application, the pharmaceutically acceptable excipient is one or more selected from the group consisting of hydroxypropyl methyl cellulose(s), polyethylene glycol(s), and nonionic surfactant(s).

In some embodiments of the present application, the pharmaceutically acceptable excipient is one or more selected from the group consisting of hydroxypropyl methyl cellulose(s), polyethylene glycol(s), and Tween(s).

In some embodiments of the present application, the orally dissolving film comprising nifedipine, ropinirole or curcumin and one or more excipients selected from the group consisting of hydroxypropyl methyl cellulose(s), polyethylene glycol(s), and Tween(s)/labrasol/glycol.

In some embodiments of the present application, the orally dissolving film comprising nifedipine, ropinirole or curcumin and one or more excipients selected from the group consisting of hydroxypropyl methyl cellulose(s), polyethylene glycol(s), and Tween(s).

In some embodiments of the present application, the orally dissolving film of the present application has an appropriate thickness, such as 5 μm-200 μm, and appropriate tensile strength and elongation percentage.

In some embodiments of the present application, the orally dissolving film of the present application comprising 3-40 wt %, preferably 5-35 wt %, more preferably 5-30 wt % of the pharmaceutically active ingredient.

In another aspect, the present application provides a method for preparing an orally dissolving film, comprising dissolving a pharmaceutically active ingredient and a pharmaceutically acceptable excipient in an organic solvent or a mixed organic solvent to obtain a solution, applying the resulting solution to form a film, and drying the film.

In some embodiments of the present application, the organic solvent includes, but is not limited to, alcohol(s), halohydrocarbon(s) (such as haloalkane(s)), acetone(s), ester(s), ether(s), derivative(s) of glycol(s) and combinations thereof.

In some embodiments of the present application, the resulting solution is coated or casted to form a film.

In another aspect, the present application provides a method for treating a disease, disorder and/or condition, comprising administering to a subject in need thereof an orally dissolving film comprising a pharmaceutically active ingredient and a pharmaceutically acceptable excipient.

In some embodiments of the present application, the orally dissolving film administered to the subject comprises nifedipine, ropinirole or curcumin and one or more excipients selected from the group consisting of hydroxypropyl methyl cellulose(s), polyethylene glycol(s), and Tween(s).

In some embodiments of the present application, the orally dissolving film is administered to the subject by an intraoral transmucosal administration route, such as sublingual administration and buccal administration.

The orally dissolving film containing ropinirole (ROP-ODF) of the present application exhibits fast disintegration and quick drug release, and is physically stable under ICH conditions for at least one month. By using the orally-dissolving film of ropinirole (ROP-ODF), the feasibility of delivering ropinirole to the systemic circulation via the oral cavity is demonstrated. The in vivo pharmacokinetic study indicates that the buccal or sublingual administration of ROP-ODF significantly improves bioavailability compared with a traditional oral administration route, which is likely attributable to avoidance of first-pass metabolism of ropinirole.

The orally dissolving film containing nifedipine (NIF-ODF) of the present application exhibits the amorphous nature as confirmed by XRPD as well as the fast disintegration and improved dissolution property of nifedipine. Upon sublingual or buccal administration, the NIF-ODF provides fast drug absorption into the systemic circulation, and significantly improves the nifedipine exposure in animals compared with a traditional oral administration route, and this may be attributable to an enhanced solubility and/or avoidance of first-pass metabolism. The NIF-ODF of the present application is also physically stable at 4° C. and relative humidity <10% for at least one month. The in-vivo pharmacokinetic study demonstrates that the NIF-ODF of the present application significantly improves the bioavailability by sublingual and buccal administration compared with oral administration of nifedipine.

The orally dissolving film containing curcumin (Curcumin-ODF) of the present application also has an increased solubility/dissolution and a fast disintegration rate in simulated saliva.

Therefore, it is clearly demonstrated the feasibility of administering a drug having low bioavailability due to the extensive first-pass metabolism and/or low water solubility upon oral administration via sublingual or buccal administration by orally dissolving films. This new dosage form is also a model for solving the application problems of water-insoluble drugs.

### Examples

Embodiments of the present application are disclosed in more detail in the following examples with reference to figures, which are not in any way intended to limit the scope of the present application.

### 1. Materials and Experimental Methods

### 1.1 Materials

Nifedipine (NIF) was a gift from Prof. Albert H. L. Chow's lab (School of Pharmacy, The Chinese University of Hong Kong, Hong Kong), and ropinirole (ROP) was purchased from Yick-Vic Chemicals & Pharmaceutics Ltd. (Hong Kong). HPMC 603 (Shin-Etsu Chemical Co. Ltd, Japan), PEG 400 and Tween 85 were gifts from Dalian Diligence Trade Co. Ltd. (Da Lian, P. R. China), BASF SE (Ludwigshafen, Germany) and Sigma (USA), respectively. Formic acid was purchased from AnalaR BDH Chemical (United Kingdom). Acetonitrile (HPLC Grade) and dichloromethane (analytical grade) were purchased from RCI Labscan Limited (Pathumwan, Thailand). Methanol (HPLC grade) was purchased from Merck Millipore (Germany). Ultrapure water was purified by a Direct-Q UV water purification system, Millipore (Germany). Tweens 85, Carbamazepine (Pharmaceutical Secondary Standard) and Trazodone Hydrochloride (HPLC grade) were purchased from Sigma-Aldrich (St. Louis, Mo., USA). All other chemicals used were of analytical grade and had a purity greater than 99.0%.

### 1.2 Preparation of ODFs

### 1.2.1 Preparation of NIF-ODF

PEG 400, Tween 85 and HPMC 603 were dissolved in Methanol:Dichloromethane=1:1, and nifedipine was subsequently added and mixed thoroughly. The final concentration of the preparation was 7.33% w/v. The resulted clear yellow solution was uniformly casted on a microscope cover glass (18×18 mm, BRAGG & Co., USA), and dried at 40° C. for 2 hours in an oven (1350FX-20 Shellab oven, Sheldon Manufacturing Inc., USA) to remove the organic solvents. The formed orally dissolving film containing nifedipine (NIF-ODF) was subsequently “peeled off” the cover glass.

### 1.2.2 Preparation of ROP-ODF

PEG 400 and HPMC 603 were dissolved in Methanol:Dichloromethane=1:1, and ropinirole was subsequently added and mixed thoroughly. The final concentration of the preparation was 8.33% w/v. The resulted clear solution was uniformly casted on a microscope cover glass (18×18 mm, BRAGG & Co., USA), and dried at 45° C. for 2 hours in an oven (1350FX-20 Shellab oven, Sheldon Manufacturing Inc., USA). Afterwards, the dried film was peeled off the cover glass using a forceps to obtain the orally dissolving film containing ropinirole (ROP-ODF).

### 1.2.3 Preparation of Curcumin-ODF

300 mg of PEG 400, 200 mg of Tween 85 and 300 mg of HPMC 603 were dissolved in Methanol:Dichloromethane=1:1, and 31 mg of curcumin was subsequently added and mixed thoroughly. The resulted solution was uniformly casted on a microscope cover glass (18×18 mm, BRAGG & Co., USA), and dried at 45° C. for 2 hours in an oven (1350FX-20 Shellab oven, Sheldon Manufacturing Inc., USA). Afterwards, the dried film was peeled off the cover glass using a forceps to obtain the orally dissolving film containing curcumin (Curcumin-ODF).

### 1.3 Characterization of ODFs

### 1.3.1 Drug Content

A piece of NIF-ODF was weighed and dissolved in 1 mL of water: acetonitrile=1:1, which was further diluted and the nifedipine content was quantified by using a UPLC.

A piece of ROP-ODF was weighed and dissolved in 1 mL of water: acetonitrile=1:1, which was also further diluted and the ropinirole content was quantified by using a UPLC.

### 1.3.2 Disintegration and Dissolution Assays

Simulated saliva was prepared according to the following recipe: 842.4 mg of sodium chloride, 1200 mg of potassium chloride, 191.1 mg of calcium chloride dihydrate, 111.6 mg of magnesium chloride hexahydrate and 348 mg of potassium phosphate dibasic were dissolved in 1 L of DI water, and the pH was adjusted to 6.8±0.05 by hydrochloric acid. Simulated saliva in a beaker was pre-warmed to 37° C. The beaker was then placed in a water bath shaker (Vision Mode: VS-1205SW1) which was kept at 37° C., and reciprocal shaking was performed at 110 rpm. An orally-dissolving film (18×18 mm, around 30 mg) was added to the simulated saliva, and disintegration time was recorded as the time for the orally dissolving film (i.e., the prepared NIF-ODF, Curcumin-ODF, and ROP-ODF) to start breaking apart (a hole is formed within the film). The simulated saliva samples were also collected from the beaker after 2 and 5 minutes to evaluate the dissolution behaviors of the orally dissolving films. The collected samples were replenished with an equal volume of fresh simulated saliva. The drug contents in the collected samples were analyzed using a UPLC

### 1.3.3 UPLC Assay

The collected samples from the drug content and dissolution studies were analyzed by using a UPLC (Agilent Technologies 1290 Infinity, USA) equipped with a Poroshell 120 EC-C18 4.6×5 mm 2.7 μm guard column and a Poroshell 120 EC-C18 4.6×100 mm 2.7 μm column at 30° C.

As for nifedipine, the mobile phase consisted of 30% of 0.1% formic acid in water and 70% acetonitrile at a flow rate at 0.5 mL/min. As for ropinirole, the mobile phase consisted of 80% of 0.1% formic acid in water and 20% acetonitrile at a flow rate of 2 mL/min.

Isocratic elution was used for the quantification of both nifedipine and ropinirole (Injection volume=5 μL), and UV detection was performed at 254 nm.

The retention time of nifedipine was about 1.657 min, and a calibration curve was constructed. It was linear in the range of 10-300 μg/mL (R²=0.9999).

The retention time of ropinirole was about 0.59 min, and the calibration curve was constructed. It was linear in the range of 12.5-400 μg/mL (R²=0.99).

### 1.3.4 DSC

DSC analysis was conducted on the neat drug (i.e., ropinirole), physical mixtures (formed from a blank film and ropinirole powder at ropinirole loadings of 5 wt %, 10 wt %, 20 wt % and 50 wt %, respectively) and ROP-ODF (ropinirole loading at 30 wt %). Thermograms were obtained by using a DSC 6000 (Perkin Elmer Instruments, USA) at a heating rate of 10° C./min from 30-280° C. under nitrogen purge at 2 kg/cm².

### 1.3.5 XPRD

A powder X-ray diffractometer (XRPD) (Xpert PRO, Hong Kong) equipped with a Cu radiation source operating at 40 kV and 40 mV was used to collect the XRPD data for different samples. Briefly, scanning from 20 of 5° to 40° at a rate of 4°/min with a step size of 0.017° was conducted on the neat drug (nifedipine), physical mixtures (formed from a blank film and nifedipine powder at nifedipine loadings of 5 wt %, 9.09 wt %, 10 wt %, and 15 wt %, respectively) and NIF-ODF (nifedipine loading at 9.09%).

### 1.3.6 FTIR

FTIR studies were conducted by using a Bruker Alpha FT-IR Spectrometer (Germany) to investigate the molecular interaction between nifedipine and other ODF excipients. A blank orally-dissolving film, nifedipine, physical mixtures of a blank film and nifedipine powder, and NIF-ODF were crushed, mixed with KBr and then compressed into pellets. FTIR spectra were collected at a resolution of >2 cm⁻¹ in the range of 500-4000 cm⁻¹.

### 1.3.7 Thickness

The thickness of ROP-ODF and that of NIF-ODF were measured by a digital vernier caliper (Mitutoyo, UK), respectively, and the reported result was the average thickness at five different locations (i.e. four corners and one at the center).

### 1.3.8 Texture Analysis

The mechanical properties of NIF-ODF and ROP-ODF were evaluated by using a TA.XT.Plus texture analyzer (Stable Micro Systems Ltd., Surrey, UK) equipped with an A/MTG load cell. Orally-dissolving films were held between two clamps positioned at 1 cm apart. The samples were pulled at 2 mm/sec. The force and elongation data were recorded when the films broke. The tensile strength and percentage of elongation were calculated accordingly.

### 1.3.9 Physical Stability

As for ropinirole-ODF, three standard ICH conditions (25° C., Relative Humidity=60%), (30° C., Relative Humidity=65%) and (40° C., Relative Humidity=75%) were set up by gas-tight jars with saturated salt solutions to achieve the desired humidity (NaCl for 40° C./75% RH, KI for 30° C./65% RH and NaBr for 25° C./60% RH). As for nifedipine-ODF, one ICH condition (25° C., Relative Humidity=60%) and a low temperature with low humidity condition (4° C., RH<10%) were set up by gas-tight jars with saturated salt solutions to achieve the desired humidity (NaBr for 25° C./60% RH and NaOH for 4° C./RH<10%).

Subsequently, the jars were placed in a Lovibond thermostatically controlled incubator (The Tintometer Ltd, Amesbury, SP4 7GR, UK) at the required temperatures. Weight gain, disintegration and dissolution were evaluated at time=0, 7 days, 14 days and 28 days according to methods described earlier.

### 1.5 In-Vivo Pharmacokinetic Study

The study followed the guidelines issued by the Department of Health, Hong Kong and the Animal Ethics Committee at the Chinese University of Hong Kong. New Zealand Albino rabbits (3.0-4.5 kg) were used in this study. The rabbits were kept at a 12/12 hour light/dark cycle with unlimited food and water supply. Before the experiments, the rabbits were fasted for 12 hours with free access to water. The rabbits were lightly anesthetized by an intramuscular injection of ketamine and xylazine immediately before dosing. In order to compensate for reduced saliva secretion due to anesthesia, the mouths of the rabbits were wetted with a small amount of distilled water (about 1 mL) before sublingual or buccal administration of the orally dissolving films.

For nifedipine, NIF-ODF (equivalent to NIF neat drug dosed at 1 mg/kg) was administered to the rabbits sublingually or buccally, and nifedipine neat drug was also dosed at 1 mg/kg orally via gastric gavage to serve as a control. Blood samples were collected periodically at pre-determined time points for 24 hours.

For ropinirole, ROP-ODF (equivalent to ROP neat drug dosed at 1.5 mg/kg) was administered to the rabbits sublingually or buccally, and ropinirole neat drug was also dosed at 1.5 mg/kg orally via gastric gavage to serve as a control. Blood samples were collected periodically at pre-determined time points for 6 hours.

### 1.6 Data Analysis

Statistical analysis was conducted by independent t-test using SPSS 19.0 to test the level of significance (p<0.05). The data points were the average of at least three trials and the error bars showed the standard deviation unless otherwise specified.

### 2. A Study on Ropinirole-Orally Dissolving Film (ROP-ODF)

### 2.1 Dissolution and Disintegration

A preliminary screening was conducted to select excipients for the formulation development of ropinirole oral films. Interestingly, various excipients and their levels impacted the disintegration and dissolution properties of ropinirole oral films minimally. Without bound to any theory, this may be attributable to the high solubility of ropinirole. A formulation consisting of 60% w/w of HPMC 603, 10% w/w of PEG 400 and 30% w/w of ropinirole was selected. The % ROP released at 2 min and 5 min were 100.0±2.5% and 100.0±2.3%, respectively, and the disintegration time was 8.7±3.6 sec.

### 2.2 Characterization of the ROP-ODF

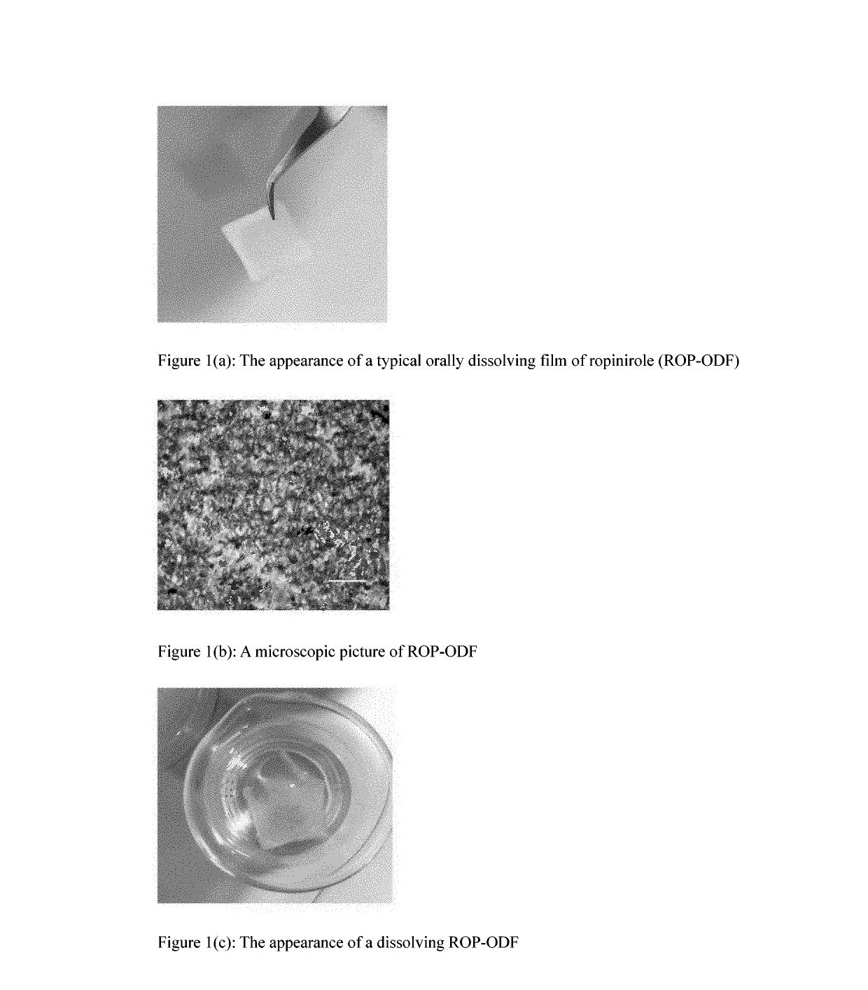

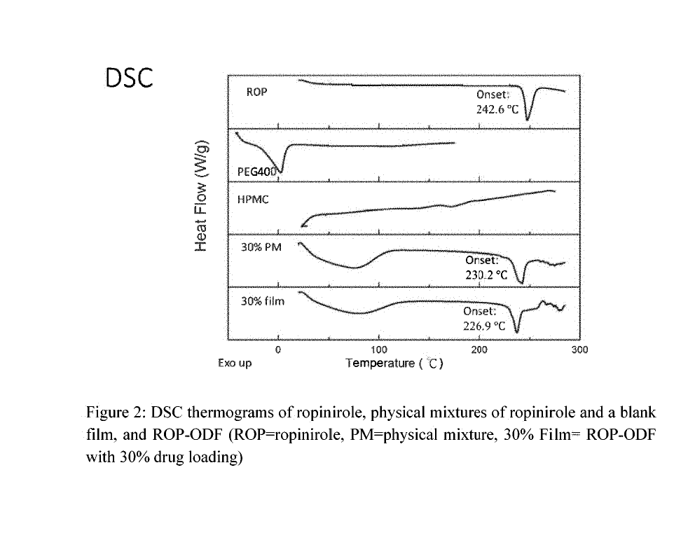

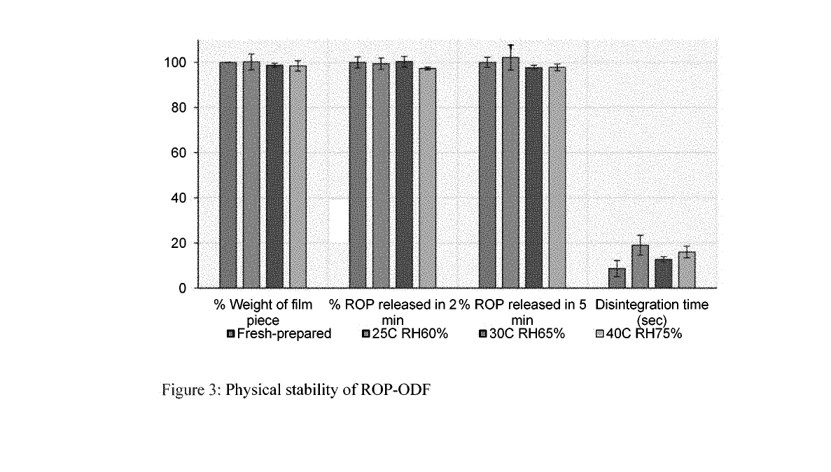

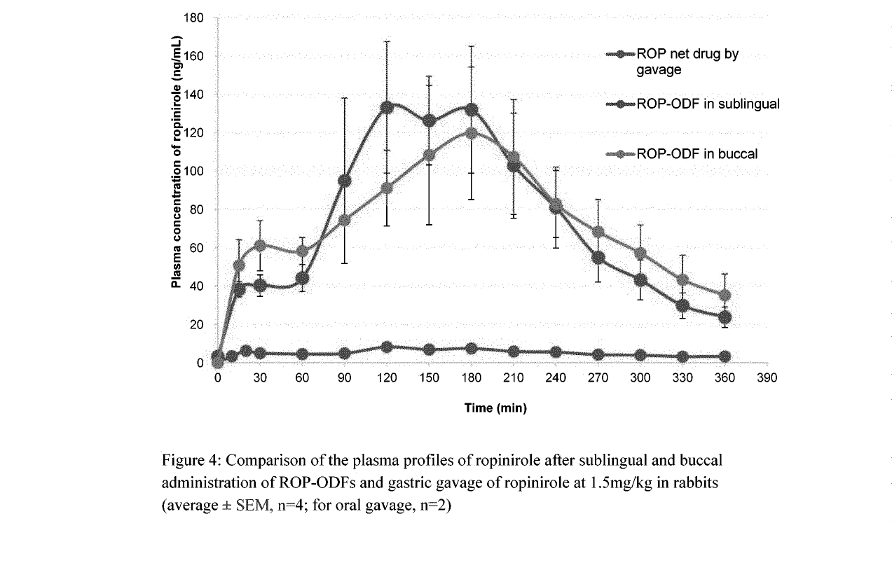

FIG. 1(a) showed the appearance of a typical ROP-ODF that appears to be off-white with opacity. This was likely related to the crystalline state of ropinirole in the film. The ROP-ODF was also examined under a polarized microscope (Nikon ECLIPSE Ti-E Microscope, Nikon, Japan), and numerous bundles of crystals were evident as shown in FIG. 1(b). The microscopic picture was in excellent agreement with the DSC thermograms, showing that an endotherm corresponding to the melting of crystalline ropinirole existed at 247.64° C. (FIG. 2). However, the crystalline state of ropinirole did not jeopardize the disintegration and dissolution properties of the orally dissolving film, and this may be related to the high water solubility of ropinirole (133 mg/mL). The appearance of a dissolving/disintegrating ROP-ODF was shown in FIG. 1(c). The ROP-ODF was fast-dissolving and complete drug release was achieved within 2 minute (Table 1). In addition, the disintegration time of ROP-ODF was about 8.7±3.6 seconds and complete disappearance of ROP-ODF was achieved within 2 minutes.

TABLE 1
Characterization of the ROP-ODF (average ± SD, n = 5; for
dissolution, n = 3)

| Characterization | ROP-ODF |
|---|---|
| Average Drug Content (%) | 99.7% |
| Content Uniformity | SD = 2.4% |
| Disintegration (sec) | 8.7 ± 3.6 |
| Dissolution at 2 min (ROP released %) | 100.0 ± 2.5 |
| Dissolution at 5 min (ROP released %) | 100.0 ± 2.3 |
| Thickness (μm) | 133.3 ± 4.5 |
| Tensile Strength (kPa) | 104.5 ± 9.9 |
| Elongation (%) | 3.3 ± 0.9 |

The average drug content of the ROP-ODFs was 99.7%, which was very close to the theoretical value (Table 1). Regarding content uniformity, the standard deviation was about 2.4%. The thickness of the ROP-ODF was determined to be 133.3±4.5 μm, which fell within a typical range for oral films (5-200 μm). All these data clearly demonstrated that exceptional robustness in the formulations and the preparation method had been achieved.

The tensile strength and % elongation of the optimized ROP-ODF were determined to be 104.5±9.9 kPa and 3.3±0.9%, respectively, which were within a reasonable range.

### 2.3 Physical Stability

The International Conference on Harmonization (ICH) provided guidelines for stability testing of new drug substances and products. The storage conditions included long-term study and accelerated study, i.e. 25° C. 60% RH, 30° C. 65% RH and 40° C. 75% RH, respectively.

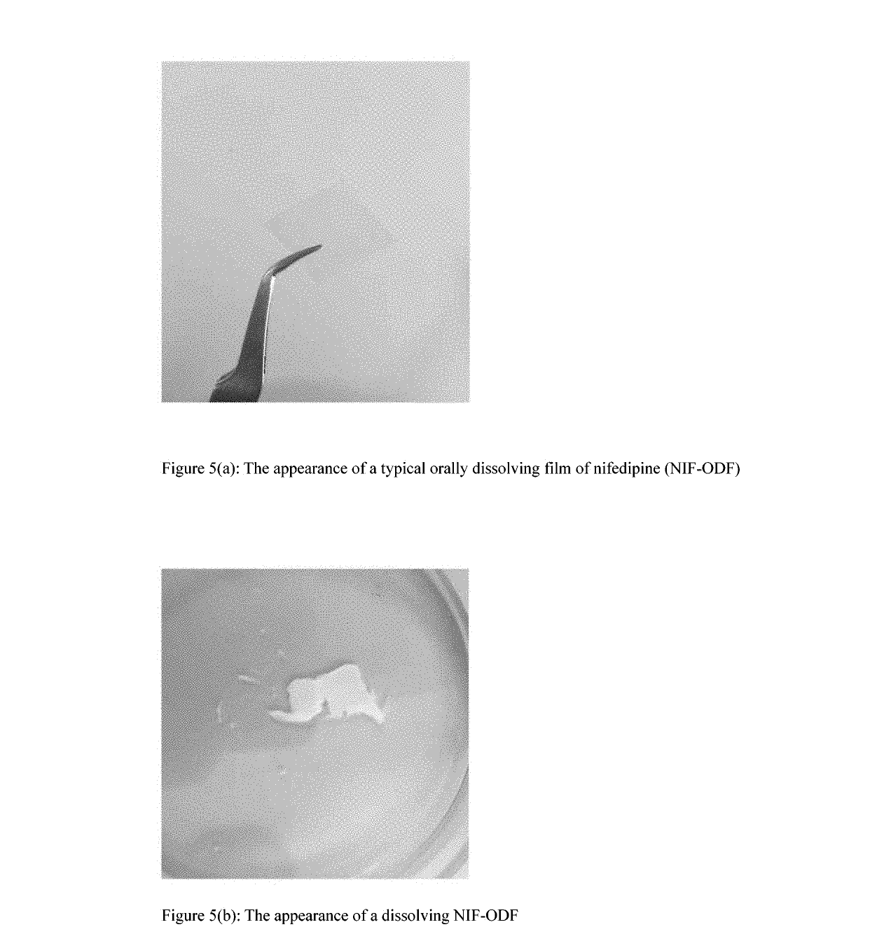

Under these conditions, the stability of the ROP-ODFs was monitored for one month and the results were shown in FIG. 3. The weight of the ROP-ODFs did not change significantly after one month of storage (paired student t-test, p>0.05), indicating that there was insignificant water sorption by the formulation upon storage.

However, the disintegration of the stored film was delayed by about 10 seconds relative to a freshly prepared film, which was determined to be statistically significant (unpaired student t-test, p<0.05). However, most importantly, the dissolution rate of the ROP-ODFs did not change significantly upon storage under the three ICH conditions (unpaired student t-test, p>0.05).

In summary, the preliminary stability data indicated that the ROP-ODF was physically stable under ICH conditions for at least one month.

### 2.4 In Vivo Pharmacokinetic Study

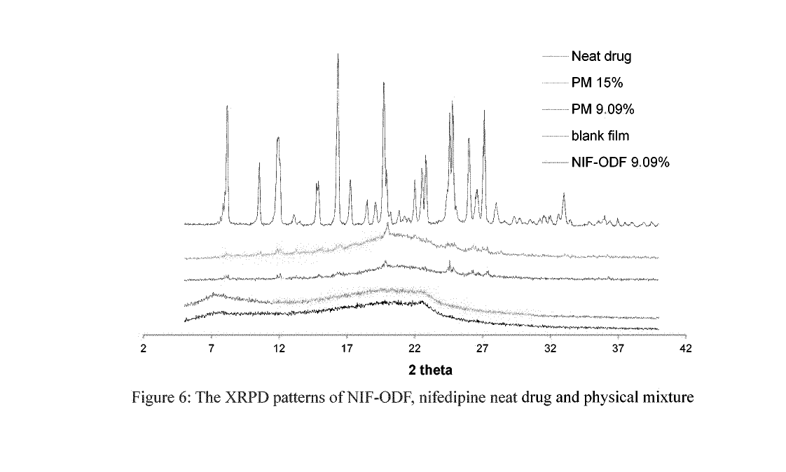

The pharmacokinetic studies were conducted by three different routes (i.e. oral, sublingual, and buccal administration), and the data were analyzed non-compartmentally by using WinNonlin (Certara USA, Inc., Princeton, N.J., USA). The plasma profiles and the pharmacokinetic parameters were shown in FIG. 4 and Table 2, respectively.

TABLE 2
Pharmacokinetic parameters of ropinirole in plasma after different
administration routes (average ± SEM, n = 4, for oral n = 2)

| Parameter/Route | Oral | Sublingual | Buccal |
|---|---|---|---|
| Tₘₐₓ (min) | 142.5 ± 53 | 170.05 ± 50.1 | 148.8 ± 16.3 |
| Cₘₐₓ (ng/mL) | 29.9 ± 16.2 | 188.9 ± 25.1 | 166.7 ± 55.2 |
| λ_{z} (min⁻¹) | 0.004 | 0.0118 ± 0.0011 | 0.0074 ± 0.001 |
| t_{1/2} (min) | 173.6 ± 1.1 | 60.2 ± 5.0 | 99.0 ± 14.4 |
| AUC_{0→6 h} | 3930.6 ± 735.3 | 30440.4 ± 2713.5 | 27634.0 ± 4843.1 |
| (ng/mL · min) | | | |
| AUC_{0→∞ (obs.,} | 5430.7 ± 533.3 | 32985.6 ± 2523.4 | 33289.6 ± 7410.9 |
| ng/mL · min) | | | |

After buccal or sublingual administration of the ROP-ODFs, ropinirole could be detected in the plasma within 15 minutes at 40 to 50 ng/mL, indicating that there was fast drug absorption into the systemic circulation.

The maximum plasma concentrations (Cₘₐₓ) achieved after the sublingual and buccal administration of the oral films were 188.9±25.1 ng/mL at 170.1±50.1 min and 166.7±55.2 ng/mL at 148.8±16.3 min, respectively.

It was remarkable to note that the bioavailability given by the sublingual or buccal route was dramatically improved compared with that after oral administration (FIG. 4). Relative to the oral administration route, the sublingually and buccally administered oral films increased the AUC_{0-6 hour} by about 7×, which was determined to be statistically significant (unpaired student t-test, p<0.05). It was important to note that only two out of four animals gave drug levels that were above the level of quantification (LOQ, 5 ng/mL) after the oral administration route. As a result, the Winolin analysis of the oral delivery data was only based on two animals. Therefore, it was likely that the improvement in the bioavailability was underestimated.

The improved bioavailability was likely related to avoidance of first-pass metabolism after buccal or sublingual administration.

The AUC (AUC_{0→6 hour} and AUC_{0→∞}) after buccal and sublingual administration of the oral films were not statistically different (unpaired t-test, p>0.05). However, buccal administration was associated with a larger inter-subject variability, and this might be related to the difficulty in applying the oral film to exactly the same buccal region in each animal.

### 3. A Study on Nifedipine-Orally Dissolving Film (NIF-ODF)

### 3.1 Dissolution and Disintegration

A preliminary screening was conducted to select excipient in the development of nifedipine oral films. It was discovered that 9.1 wt % drug loading was very close to the maximum drug loading in which nifedipine can remain as amorphous in the film. In addition, 45.5 wt % of HPMC 603 and 9.0 wt % of PEG 400 were the levels that provided sufficient mechanical strength and flexibility for the film to be manufactured and handled. As a result, nifedipine oral film formulation consisting of 45.5 wt % of HPMC 603, 9.0 wt % of PEG 400, 36.4 wt % of Tweens 85 and 9.1 wt % of nifedipine was selected and this formulation was used for further study. The observed percentages of released NIF at 2 min and 5 min were 64.0±6.88% and 65.4±7.48%, respectively. Besides, the disintegration time of selected NIF-ODF was detected as 11.6±2.3 sec.

### 3.2 Characterization of the Oral Film

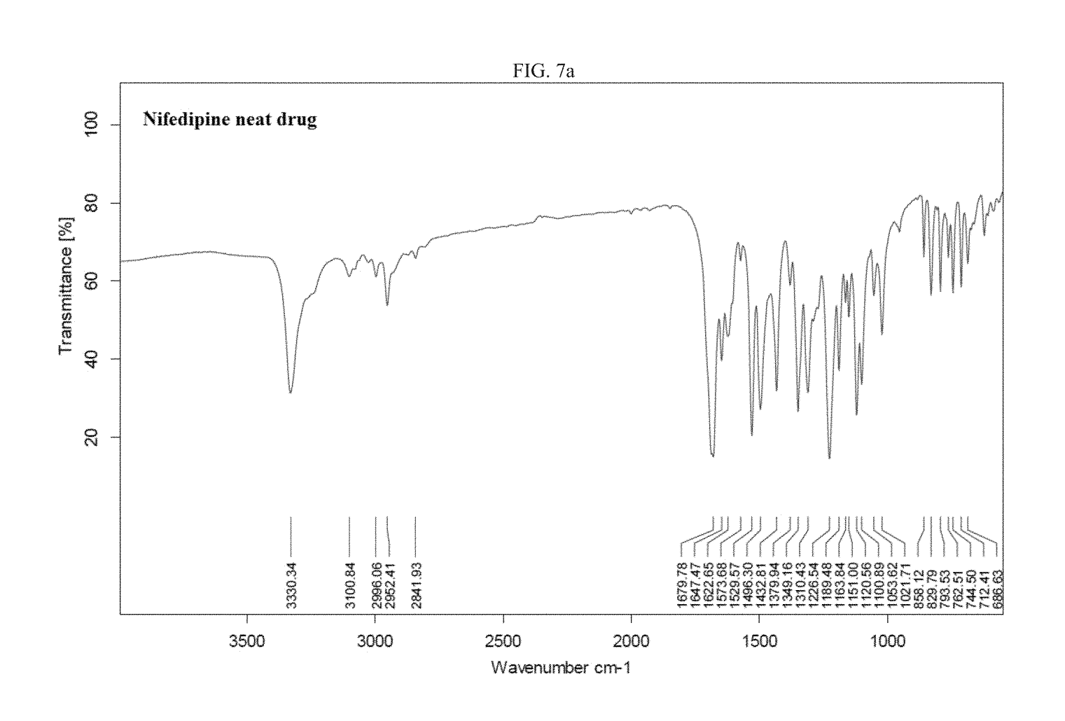

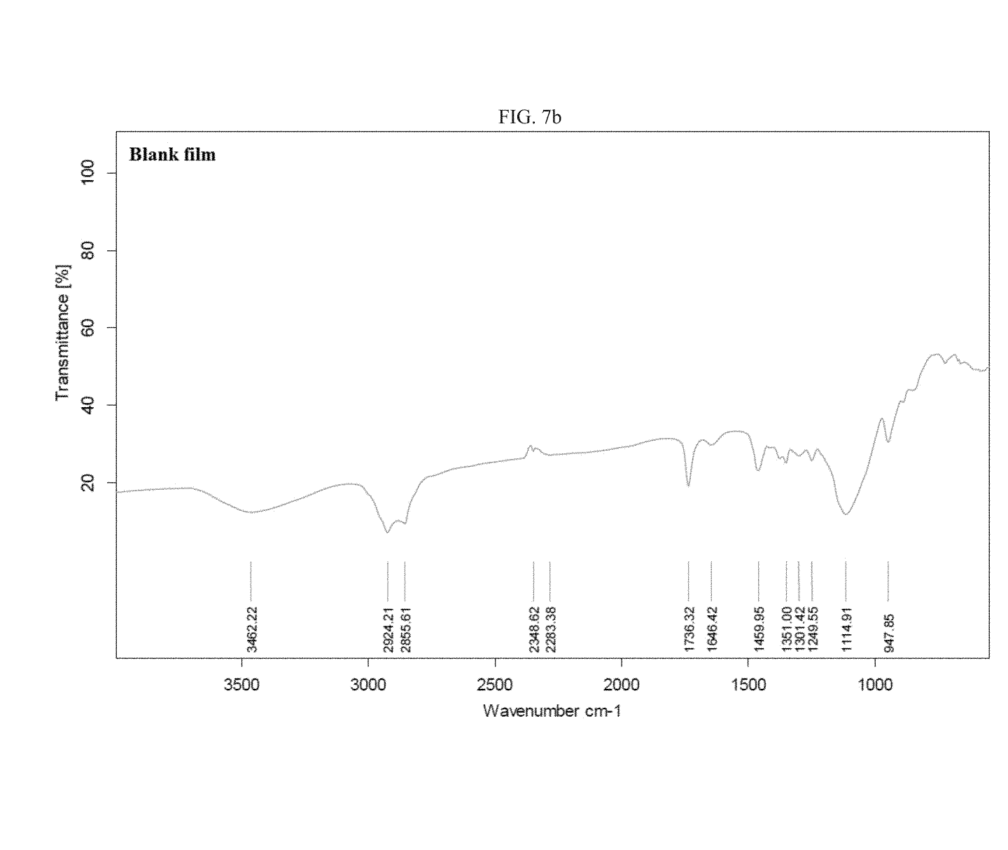

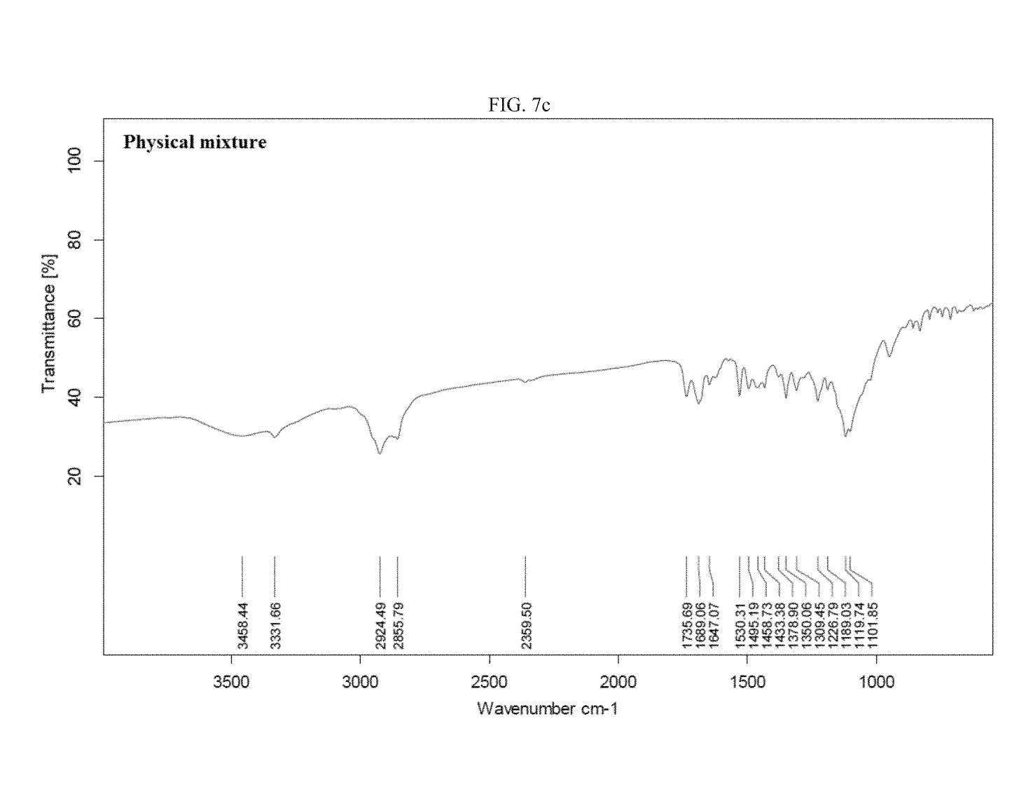

The appearance of a typical nifedipine oral film was shown in FIG. 5(a). It appeared to be transparent with a yellowish color. The transparency may imply that nifedipine existed as an amorphous state in the film, which was consistent with the XRPD spectrums (FIG. 6) in which only a “halo” pattern was observed without any crystalline peak.

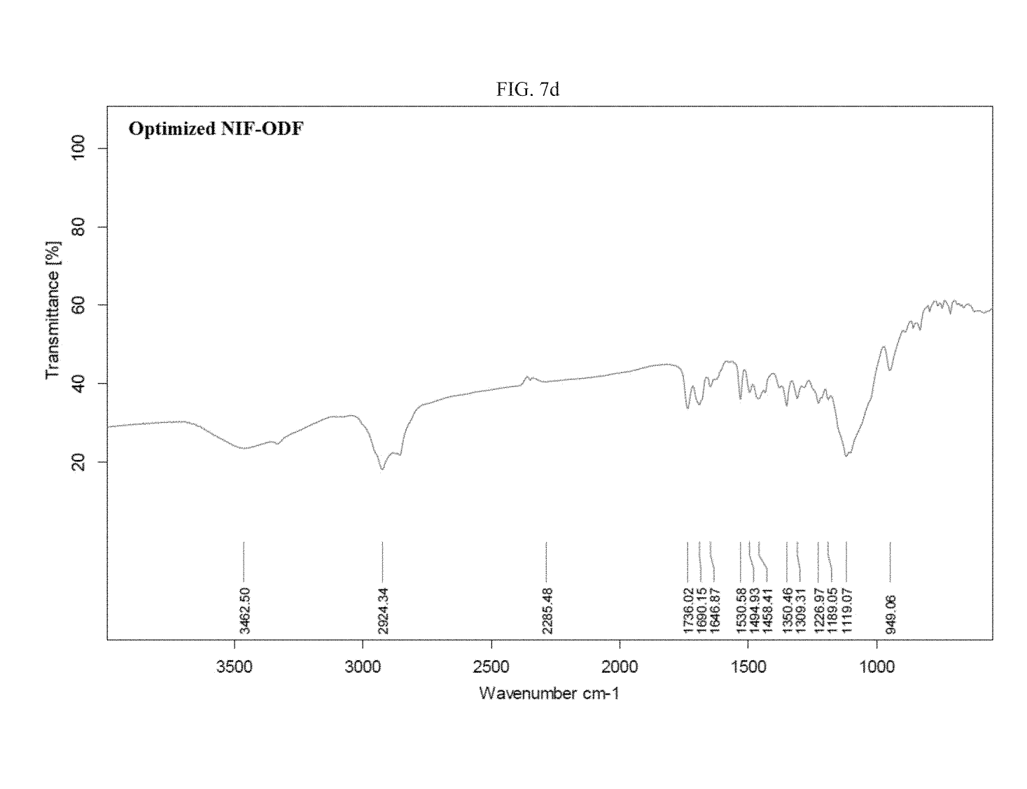

Although nifedipine appeared to be amorphous in the film, significant molecular interactions between nifedipine and other components in the film did not exist, as elucidated in the FTIR spectra as shown in FIG. 7. The N—H stretching peak 3330 cm⁻¹ and the sharp NO₂ stretching peak 1530 cm⁻¹ were observed in both the neat drug and the physical mixture, and these peaks were not altered in the nifedipine oral film, indicating that hydrogen bondings were not formed between the drug and the excipients. Indeed, the FTIR spectra between the physical mixture and the nifedipine were essentially the same.

The average drug content of the nifedipine oral film was determined to be 101.6%, which was very close to the theoretical value. A consistent content uniformity (sd=0.02312) was also achieved (Table 3). The thickness of the nifedipine oral film was measured as 140±10 μm, which fell within a typical range for oral films (5-200 μm). The consistent drug content and content uniformity results indicated exceptional robustness of the formulation and manufacturing process had been achieved.

A complete drug release was not achieved at 2 minute and only 64.0±6.88% of the loaded drug was released. The solubility of nifedipine at pH 7 was about 6 μg/mL. The oral film increased the solubility substantially by about 10× to about 59.0±5.82 μg/mL at 2 minute. The drug concentration achieved at 5 minutes was 60.20±6.11 μg/mL, which was comparable to that achieved at 2 minutes. Without bound to any theory, the improved solubility was likely attributable to the formation of a supersaturated drug solution because of the amorphous nature of the incorporated drug. The disintegration time of the film was determined to be 11.6±2.3 seconds. Typically, disintegration was completed within 2 minutes.

The measured tensile strength of the nifedipine oral film was 46.6±3.1 kPa, and the % elongation was 4.6±0.6%.

TABLE 3
Characterization of the orally-dissolving film of nifedipine
(average ± SD, n = 5; for dissolution, n = 3).

| | Nifedipine Oral |
|---|---|
| Characterization | Film |
| Uniformity of drug content (%) | 101.6 ± 0.02312 |
| Disintegration (sec) | 11.6 ± 2.3 |
| Dissolution at 2 min (% released) | 64.0 ± 6.88 |
| Dissolution at 5 min (% released) | 65.4 ± 7.48 |
| Thickness (μm) | 140 ± 10 |
| Tensile Strength (kPa) | 46.6 ± 3.10 |
| Elongation (%) | 4.6 ± 0.60 |

### 3.3 Physical Stability

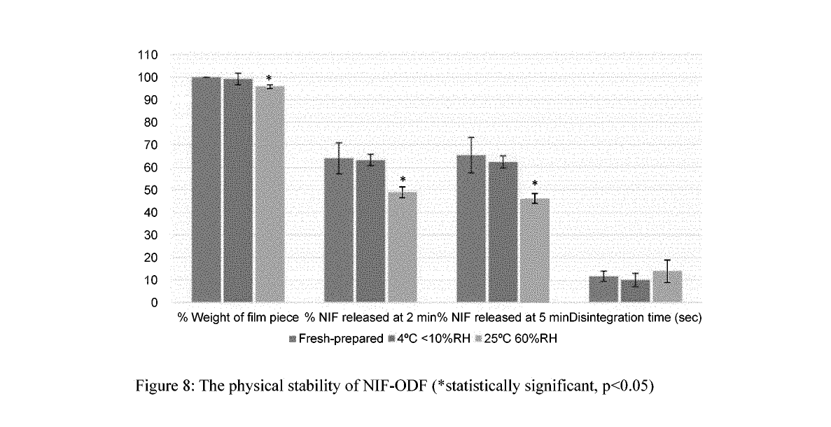

The stability of NIF-ODF was detected for one month under two storage conditions, i.e. 25° C. 60% RH and 4° C.<10% RH respectively, and the physical stability results of the nifedipine oral film were shown in FIG. 8. It was obvious that the nifedipine oral film did not show significant change in its weight, dissolution rate and disintegration time (paired student t-test for weight gain, unpaired student t-test for other, p>0.05) under the 4° C./RH<10% condition after 28 days. Under the condition of 25° C./60% RH, the oral film exhibited a statistically significant decrease in the dissolution rate (unpaired student t-test, p<0.05) while the disintegration time remained unchanged (unpaired student t-test, p>0.05), which indicated that nifedipine might crystallize out from the film when it was exposed to room condition after one month. This implied that nifedipine ODF may need to store under low temperature and low humidity conditions.

### 3.4 In Vivo Pharmacokinetic Study

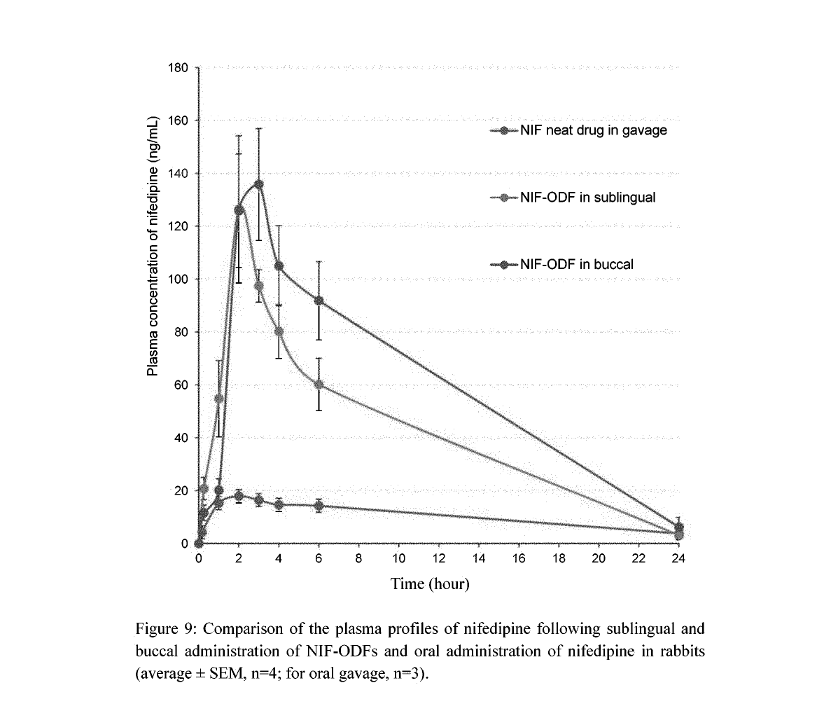

FIG. 9 showed the results of the pharmacokinetic studies after drug administration using three different routes (i.e. oral, sublingual and buccal). The pharmacokinetic data were analyzed non-compartmentally by using WinNonlin (Certara USA, Inc., Princeton, N.J., USA) and the results were shown in Table 4.

TABLE 4
The Winolin analysis of the pharmacokinetic data
(average ± SEM, n = 4; for oral gavage, n = 3).

| Parameter/ | Neat drug | Oral Film | |
|---|---|---|---|
| Route | Gavage | Sublingual | Buccal |
| Tₘₐₓ (min) | 141.3 ± 11.6 | 163.8 ± 27.8 | 130.0 ± 11.5 |
| Cₘₐₓ (ng/mL) | 19.2 ± 3.7 | 158.8 ± 37.1 | 176.7 ± 9.1 |
| λ_{z} (min⁻¹) | 0.0011 ± 0.0002 | 0.0024 ± 0.0002 | 0.0029 ± 0.0005 |
| t_{1/2} (min) | 681.7 ± 100.8 | 290.6 ± 21.3 | 257.7 ± 37.2 |
| AUC_{0→24 h} | 13538.5 ± 1826.6 | 52310.8 ± 5929.4 | 63702.4 ± 5254.3 |
| (ng/mL · min) | | | |
| AUC_{0→∞} | 17295.0 ± 1277.2 | 54299.6 ± 5768.6 | 65953.4 ± 6143.2 |
| (obs., | | | |
| ng/mL · min) | | | |

These three extravascular administration routes gave similar Tₘₐₓ of 2-3 hours. Oral administration of nifedipine neat drug via oral gavage achieved the lowest drug level and bioavailability (Cₘₐₓ=19.2±3.7 ng/mL, AUC_{0→24h}=13538.5±1826.6 ng/mL·min and AUC_{0→∞} (obs.)=17295.0±1277.2 ng/mL·min). This is not unexpected because of the poor solubility and extensive first-pass metabolism of nifedipine as reported in the research literatures.

After sublingual or buccal administration of the oral film, nifedipine was able to be detected in the plasma within 15 minutes, indicating that there was fast drug absorption into the systemic circulation. Sublingual administration of nifedipine oral film achieved 20.78 ng/mL plasma level at 15 minute and the Cₘₐₓ was about 158.8±37.1 ng/mL at 163.8±27.8 minutes. After buccal administration of the nifedipine oral film, the drug plasma level reached 11.6±2.9 ng/mL at 15 minutes and the Cₘₐₓ of 176.7±9.1 ng/mL was achieved at 130.0±11.5 min.

Typically, for a sublingual or buccal administration of the drug, the existence of a second peak after Cₘₐₓ may imply that the administered drug is swallowed which is subsequently absorbed through the gastrointestinal tract. However, this is indeed not the case as only one peak was observed in the plasma-time profiles of both buccally and sublingually administered oral films. This may indirectly indicate that a minimal amount of nifedipine was swallowed. However, seeing that the drug level obtained after the oral administration of nifedipine was extremely low (FIG. 9), the absorption of the swallowed nifedipine through the gastrointestinal tract might not be able to give rise to a second peak in the plasma-time profiles.

Relative to a traditional oral administration route, both the buccal and sublingual administration routes significantly improved the Cₘₐₓ, AUC_{0→24h} and AUC_{0→∞} by 3.9-4.7×, and 3.0-3.8×, respectively, which were determined to be statistically significant (unpaired student t-test, p<0.05). This may be attributable to an enhanced drug solubility and avoidance of first-pass metabolism.

The pharmacokinetic data (Cₘₐₓ, AUC_{0→24h} and AUC_{0→∞}) obtained after sublingual and buccal administration were comparable without any statistically significant difference (unpaired student t-test, p>0.05), which was in good agreement with our separate study employing ropinirole as a model compound. Showing similar results to the ropinirole study, the inter-subject variability associated with the buccal administration was higher than that after the sublingual administration of the oral films (FIG. 9), and this may be attributable to the difficulty in applying the oral film to the same buccal area in each animal.

In summary, sublingually or buccally administered nifedipine oral film achieved fast drug level in the systemic circulation and significantly improved the bioavailability relative to the oral administration route.

What is claimed is:

1. An orally dissolving film comprising a pharmaceutically active ingredient and a pharmaceutically acceptable excipient.

2. The orally dissolving film of claim 1, wherein the pharmaceutically active ingredient has a low bioavailability due to first-pass metabolism and/or low water solubility upon oral administration.

3. The orally dissolving film of claim 2, wherein the pharmaceutically active ingredient is selected from the group consisting of nifedipine, curcumin and ropinirole.

4. The orally dissolving film of claim 1, wherein the pharmaceutically active ingredient is water-soluble or water-insoluble, and has a low bioavailability due to first-pass metabolism and/or low water solubility upon oral administration.

5. The orally dissolving film of claim 1, wherein the pharmaceutically active ingredient is amorphous, partially crystalline or crystalline.

6. The orally dissolving film of claim 1, wherein the pharmaceutically acceptable excipient is one or more selected from the group consisting of a film-forming material(s), adhesive(s), lubricant(s), surfactant(s), plasticizer(s), filler(s), coloring agent(s) and release agent(s).

7. The orally dissolving film of claim 6, wherein the pharmaceutically acceptable excipient is one or more selected from the group consisting of polyvinyl alcohol(s), ethylene-vinyl acetate copolymer(s), acrylic copolymer(s) (such as methacrylate-methylacrylic acid copolymer), cellulose(s) and derivative(s) thereof (such as hydroxypropyl methyl cellulose, hydroxypropyl cellulose, methylcellulose, ethylcellulose and the like), polyvinylpyrrolidone(s), silicon rubber(s), polylactic acid(s), polyethylene glycol(s), surfactant(s), plasticizer(s), filler(s), coloring agent(s), and release agent(s).

8. The orally dissolving film of claim 7, wherein the pharmaceutically acceptable excipient is one or more selected from the group consisting of cellulose(s) and derivative(s) thereof, polyethylene glycol(s), and surfactant(s).

9. The orally dissolving film of claim 8, wherein the pharmaceutically acceptable excipient is one or more selected from the group consisting of hydroxypropyl methyl cellulose(s), polyethylene glycol(s), and nonionic surfactant(s).

10. The orally dissolving film of claim 9, wherein the pharmaceutically acceptable excipient is one or more selected from the group consisting of hydroxypropyl methyl cellulose(s), polyethylene glycol(s), and Tween(s).

11. The orally dissolving film of claim 1, comprising nifedipine, curcumin or ropinirole, and one or more excipients selected from the group consisting of hydroxypropyl methyl cellulose(s), polyethylene glycol(s), and Tween(s).

12. The orally dissolving film of claim 1, comprising nifedipine and hydroxypropyl methyl cellulose(s), polyethylene glycol(s), and Tween(s), or comprising ropinirole, hydroxypropyl methyl cellulose(s), and polyethylene glycol(s), or comprising curcumin and hydroxypropyl methyl cellulose(s), polyethylene glycol(s), and Tween(s).

13. The orally dissolving film of claim 1, wherein the orally dissolving film has a thickness of 5 μm-200 μm.

14. A method for preparing an orally dissolving film of claim 1, comprising dissolving a pharmaceutically active ingredient and a pharmaceutically acceptable excipient in an organic solvent or a mixed organic solvent to obtain a solution;
applying the resulting solution to form a film; and
drying the film.

15. The method of claim 14, wherein the organic solvent includes an alcohol(s), halohydrocarbon(s) or combinations thereof.

16. The method of claim 14, wherein the resulting solution is coated or casted to form a film.

17. A method for treating a disease, disorder and/or condition, comprising administering to a subject in need thereof an orally dissolving film of claim 1.

18. The method of claim 17, wherein the orally dissolving film is administered to the subject by an intraoral transmucosal administration route.

19. The method of claim 18, wherein the intraoral transmucosal administration route is sublingual administration.

20. The method of claim 18, wherein the intraoral transmucosal administration route is buccal administration.
